(19) United States
(12) Patent Application Publication
(10) Pub. No.: US 2025/0057810 A1
(43) Pub. Date: Feb. 20, 2025

(54) AQUEOUS MELATONIN FORMULATION

(71) Applicant: AGB-Pharma AB, Lund (SE)

(72) Inventor: Staffan WAXEGÅRD, Lund (SE)

(21) Appl. No.: 18/722,045

(22) PCT Filed: Dec. 20, 2022
(86) PCT No.: PCT/EP2022/086992

§ 371 Date: Jun. 20, 2024

(30) Foreign Application Priority Data
Dec. 20, 2021 (EP) 21216042.8

(51) Int. Cl. A61K 31/4045; A61K 47/10; A61K 47/26

(52) U.S. Cl. CPC A61K 31/4045; A61K 47/10; A61K 47/26

(57) ABSTRACT

The present invention relates to an aqueous melatonin formulation which is devoid of unnecessary excipients and potentially harmful substances. In particular, the present invention relates to an aqueous melatonin composition that comprises potassium sorbate as the only preservative.

### TECHNICAL FIELD OF THE INVENTION

The present invention relates to an aqueous melatonin formulation which is devoid of unnecessary excipients and potentially harmful substances. In particular, the present invention relates to an aqueous melatonin composition that comprises potassium sorbate as the only preservative.

### BACKGROUND OF THE INVENTION

Sleep disorders affect hundreds of millions of people worldwide on a continuous basis, many of which suffer from negative impact on quality of life, lack of productivity and high health care utilization. The most prevalent sleep disorder is insomnia or more literally the inability to sleep. Insomnia has various etiologies and degrees of severeness and is estimated to affect up to 6-12% of the adult population and as much as between 15-25% of children. Accordingly, there is a massive need for providing treatment of sleep disorders.

Traditionally, synthetic hypnotics such as barbiturate and later benzodiazepines and quinazolinones have been used to induce sleep. Predominantly, benzodiazepines enhancing the effect of the neurotransmitter gamma-aminobutyric acid (GABA) at the GABAA receptor have been prescribed to individuals suffering from a sleep disorder. However, long-term use of benzodiazepines is not recommended due to the risk of dependence, daytime sedation and respiratory depression, and at best only intermittent administration at the lowest effective dose is commended.

Benzodiazepines also generally worsen sleep quality by increasing light sleep and decreasing deep sleep.

In response to the safety concerns revolving benzodiazepines, non-benzodiazepine hypnotics (or so-called Z-drugs) were introduced in the early 1990's. These drugs are “benzodiazepine-like” in nature and display similar pharmacodynamic profiles. Although the use of non-benzodiazepines has become more popular, the alleged improved efficacy is still debated and side-effects similar to those of benzodiazepines are frequent.

In contrast to the synthetic hypnotics, melatonin is a naturally occurring indole hormone released by the pineal gland located in the brain. It is released primarily at night and it is well-established that melatonin is involved in modulation of the circadian rhythm regulating the sleep-wake cycle, asserting its effect predominantly through its interaction with the melatonin receptors. Melatonin has also been found to modulate sleep patterns related to seasonal cycles.

Melatonin is a neurohormone with the chemical identity N-acetyl-5-methoxytryptamine and is made from tryptophan via serotonin as an intermediate. In the past, melatonin was derived from bovine pineal tissue, but today it is mainly synthetic, which limits the risk of contamination or the means of transmitting infectious material.

Use of melatonin to induce sleep has several advantages over conventional synthetic hypnotics. First, melatonin is naturally metabolized with blood levels returning to normal daytime levels within 6-8 hours of administration. Therefore, melatonin does not cause adverse effects the day following administration as is in many instances an issue with conventional hypnotics. Importantly, melatonin does not present the same risk of dependency as conventional hypnotics and being a naturally occurring and endogenous compound does not induce amnesia effects as benzodiazepines.

Given the many benefits of melatonin as a sleep-inducing agent its use as pharmaceutical has been widely explored. Melatonin may be provided in either solid dosage forms or in liquid dosage forms. For consumers that have difficulties swallowing pills or tablets, such as children or individuals suffering from dysphagia, liquid dosage form is preferred.

Unfortunately, liquid dosage forms have shorter shelf-life than solid dosage forms. To overcome this drawback, excipients such as preservatives and/or antioxidants are often added to liquid formulations to extend their shelf-life. Typically, these excipients are used in combination to achieve a satisfactory preservatory effect. However, many excipients pose a health risk to the consumer, and limitation of unnecessary excipients is therefore advantageous.

Plenty of examples of adverse effects of excipients have been documented. Parabens, such as methyl-, ethyl-, propyl-, and butylparaben, are suspected to cause allergy, endocrine disruption, and cancer. This have let to the ban of parabens in some products, and especially in products directed at children. Benzoates, such as sodium benzoate, have been shown to cause hypersensitivity in individuals suffering from asthma, and has also been linked to hyperactivity in children. Citric acid (E330) is a frequently used antioxidant which have been linked to kidney disease and can for some individuals cause mouth irritation or an upset stomach. Thus, if possible, it is preferable to avoid liquid dosage forms without excessive numbers of excipients.

Hence, it would be advantageous to provide a liquid melatonin formulation which is devoid of unnecessary excipients and potentially harmful substances. Specifically, aqueous melatonin compositions comprising only a single preservative would be advantageous for use with vulnerable individuals, such as children and adolescents.

### SUMMARY OF THE INVENTION

Formulations for mitigating sleep disorders is of great importance when put in perspective of how many lives are affected every single day. The naturally occurring neurohormone melatonin may be used to induce sleep without the severe risk of adverse effects associated with conventional synthetic hypnotics. Unfortunately, liquid formulations of melatonin in many cases contain unhealthy excipients, such as excess preservatives, or allergenic substances. Thus, there is an unmet need to provide a liquid melatonin composition that is stripped from unnecessary and harmful substances.

The present invention relates to the provision of a liquid melatonin formulation that comprises a minimum of components. In particular, it has been found that storage stability can be retained by inclusion of a single and non-harmful preservative.

Thus, an object of the present invention relates to the provision of a liquid melatonin formulation that is suitable for use with children, adolescents and individuals that react adversely to one or more excipients contained in similar products.

Thus, an aspect of the present invention relates to an aqueous melatonin composition comprising:
i) an active ingredient selected from melatonin, and
ii) a preservative selected from potassium sorbate, dissolved in an aqueous phase,
   wherein the aqueous phase comprises at least about 85% w/v water, and wherein the composition is free of any additional preservatives.

Another aspect of the present invention relates to an aqueous melatonin composition as described herein for use as a medicament.

Yet another aspect of the present invention relates to an aqueous melatonin composition as described herein for use in treatment of sleep disorders.

A further aspect of the present invention relates to a method for preparing an aqueous melatonin composition as described herein, said method comprising the steps of:
i) Provision of an aqueous solvent,
ii) Addition of potassium sorbate and melatonin to the aqueous solvent to obtain an aqueous melatonin solution,
iii) Adjustment of the pH of said aqueous melatonin solution,
iv) Filtration of said aqueous melatonin solution, thereby obtaining said aqueous melatonin composition.

A still further aspect of the present invention relates to an aqueous melatonin composition as described herein obtained by a method as described herein.

The present invention will in the following be described in more detail.

### DETAILED DESCRIPTION OF THE INVENTION

### Definitions

Prior to outlining the present invention in more details, a set of terms and conventions is first defined:

### Melatonin

In the present context, the term “melatonin” refers to the neurohormone released by the pineal gland in the brain. Melatonin has the chemical identity N-acetyl-5-methoxytrypamine. One form of melatonin is micronized melatonin, wherein the melatonin particles have been comminuted to decrease particle size.

### Micronized Melatonin

In the present context, the term “micronized melatonin” refers to a population of melatonin particles with a D90 of 30 μm or less when measured using laser diffraction. D90 describes the value at which 90% of the particles within the population have a diameter below this value.

Particle size and particle size distribution (PSD) can be determined using a Malvern Mastersizer 2000 from Malvern Instruments with a measuring cell Hydro 2000pP. This particle size analyzer is based on laser diffraction and can accurately determine the PSD of solid particulate melatonin. Low angle laser light scattering is responsive to the volume of a particle and yields a volume-average particle size, which is equivalent to the weight-average particle size as the density is held constant.

Settings of the Malvern Mastersizer 2000 (Hydro 2000S measuring cell) were as follows.

Analysis model: General purpose (Particle shape=irregular)

Optical properties material: RI=1.60 absorption=0

Pump speed: 2500 rpm

Ultrasonics: None

Dispersant name: Heptane, heptane RI: 1.39

Melatonine F RI: 1.6 Absorption=0

Sample quantity: 50 mg

Obscuration: 5-25%

Measurements time: 60 seconds

Background time: 10 seconds

Aliquots: 1 for SOP

Measurements: 3 for aliquot

Delay: 10 seconds

Create average results: yes

Clean mode: Manual full wash

### Preservative

In the present context, the term “preservative” refers to a substance or chemical that prevent decomposition of the aqueous melatonin composition by microbial growth or by unwanted chemical changes.

One group of preservatives are antimicrobial preservatives, which specifically prevent degradation by bacteria. Antimicrobial preservatives include, but are not limited to, benzoates, parabens, sorbates and isothiazolines.

### Antioxidant

In the present context, the term “antioxidant” refers to a substance or chemical that prevent or inhibit the oxidation process. Antioxidants include also sequestering agents, such as citric acid, which collect metal ions that would otherwise catalyse the oxidation process.

### Active Ingredient

In the present context, the term “active ingredient” refers to any ingredient in a composition that is biologically or pharmacologically active. The term “active ingredient” encompasses both “active pharmaceutical ingredient (API)” as typically used for medicines and “active substance” as sometimes used for natural products.

Non-active ingredients are referred to herein as excipients.

### Alcohol

In the present context, the term “alcohol” refers to any organic compound that comprises one or more hydroxyl (OH) functional groups bound to a saturated carbon atom.

Alcohols include, but are not limited to, organic compounds with only a single hydroxyl group (monohydric alcohols), organic compounds with multiple hydroxyl groups (polyhydric alcohols), and cyclic alcohols.

### Flavouring Agent

In the present context, the term “flavouring agent” refers to a substance that is used to disguise any unpleasant taste caused by an active ingredient or other ingredients and improve the organoleptic experience of the consumer. Flavouring agents may be of natural or artificial origin.

### Storage Stability

In the present context, the term “storage stability” refers to the minimum period of time a solution can be stored without spoilage from microbial contamination in accordance with European Pharmacopoeia 10^{th} Edition, 5.1.3 Efficacy of antimicrobial preservation (Ph.Eur.5.1.3).

The stability is assessed by challenging the solution, such as the compositions described herein, with a defined set of microorganisms and measure the proliferation of the microorganisms over a period of 4 weeks.

Storage stability upon more extended storage periods can be assessed by storing the composition and withdrawing samples for measurement of microbial presence at various time points, such as after 3 months, 6 months, 1 year and 2 years.

### About

Wherever the term “about” is employed herein in the context of amounts, for example absolute amounts, such as numbers, purities, weights, sizes, etc., or relative amounts (e.g. percentages, equivalents or ratios), timeframes, and parameters such as temperatures, pressure, etc., it will be appreciated that such variables are approximate and as such may vary by +10%, for example #5% and preferably #2% (e.g. #1%) from the actual numbers specified. This is the case even if such numbers are presented as percentages in the first place (for example ‘about 10%’ may mean+10% about the number 10, which is anything between 9% and 11%).

### Melatonin Formulation in Solid Dosage Form and Method for the Production Thereof

Sleep disorders affect millions of individuals worldwide every single day. Synthetic means for mitigating the negative impact of sleep disorders have existed for decades. Unfortunately, synthetic hypnotics, such as barbiturate, benzodiazepines and benzodiazepine-like compounds, have questionably safety profiles and efficacy has not been acknowledged for all compounds.

Melatonin is a naturally occurring neurohormone that is an alternative active ingredient to combat sleep disorders that does not cause the severe adverse effects associated with the synthetic counterparts. Yet, many melatonin compositions are provided as liquid solutions with a range of preservatives that have proven to cause significant short-term and long-term adverse effects, such as endocrine disruption, allergy or eczema. Especially, combinations of excipients are utilized to obtain long shelf-life, with the clear drawback that the increased number of compounds in a formulation increases the risk of adverse effects. Accordingly, there is an unmet need to provide melatonin as a health-friendly composition without any unnecessary excipients.

Herein is provided an aqueous melatonin composition comprising only a single preservative, namely potassium sorbate. It has been found that a good preservatory effect can be achieved by using potassium sorbate alone as tested in accordance with Ph. Eur. 5.1.3. Moreover, it was found that potassium sorbate displayed favourable organoleptic properties. The composition is safe and can be stored for extended periods of time.

Thus, an aspect of the present invention relates to an aqueous melatonin composition comprising:
i) an active ingredient selected from melatonin, and
ii) a preservative selected from potassium sorbate, dissolved in an aqueous phase,
   wherein the aqueous phase comprises at least 85% w/v water, and
   wherein the composition is free of any additional preservatives.

The active ingredient, melatonin, may preferably be provided in micronized form. Melatonin in micronized form differs from traditional melatonin in that it is processed to particles of smaller size. This processing impacts particle characteristics such as shape, size and size distribution. Thus, micronized melatonin particles are typically smaller than about 30 μm. Melatonin in micronized form is preferred as it reduces the time needed to dissolve the melatonin during preparation of the aqueous composition. Since melatonin is added under rigorous stirring at high speed it is desired to make this step as efficient as possible.

Therefore, an embodiment of the present invention relates to the aqueous melatonin composition as described herein, wherein the melatonin is in the form of micronized melatonin.

Another embodiment of the present invention relates to the aqueous melatonin composition as described herein, wherein the size of the micronized melatonin (D90) is in the range of about 5 μm to about 20 μm, such as about 5 μm to about 10 μm.

A further embodiment of the present invention relates to the aqueous melatonin composition as described herein, wherein the micronized melatonin has a D90 value of about 30 μm or less, preferably about 20 μm or less, more preferably about 10 μm or less.

The concentration of melatonin is selected so that that the active ingredient can be administered in doses of suitable sizes to a variety of individuals, e.g. children, adolescents and adults. The remaining ingredients of aqueous melatonin composition is generally inactive, i.e. they do not contribute significantly to the sleep-inducing effect.

Therefore, an embodiment of the present invention relates to the aqueous melatonin composition as described herein, wherein the concentration of melatonin is in the range of about 0.5 mg/ml to about 2.0 mg/ml, such as about 0.75 mg/ml to about 1.5 mg/ml, such as about 0.9 mg/ml to about 1.1 mg/ml, preferably about 1 mg/ml.

Another embodiment of the present invention relates to the aqueous melatonin composition as described herein, wherein said composition is free of additional active ingredients.

A further embodiment of the present invention relates to the aqueous melatonin composition as described herein, wherein said additional active ingredients are active pharmaceutical ingredients (APIs).

The good preservatory effect observed for potassium sorbate does not require unusually high concentration of preservative compared to commercially available liquid formulations of melatonin comprising preservatives different from potassium sorbate or potassium sorbate in combination with an additional preservative.

Thus, an embodiment of the present invention relates to the aqueous melatonin composition as described herein, wherein the concentration of potassium sorbate is less than about 2.0 mg/ml, such as less than about 1.5 mg/ml, such as less than about 1.2 mg/ml.

Another embodiment of the present invention relates to the aqueous melatonin composition as described herein, wherein the concentration of potassium sorbate is in the range of about 0.5 mg/ml to about 2.0 mg/ml, such as about 0.75 mg/ml to about 1.5 mg/ml, such as about 0.9 mg/ml to about 1.1 mg/ml, preferably about 1 mg/ml.

A further embodiment of the present invention relates to the aqueous melatonin composition as described herein, wherein said composition comprises;
i) about 0.5 mg/ml to about 2.0 mg/ml melatonin, preferably about 1 mg/ml melatonin, and
ii) about 0.5 mg/ml to about 2.0 mg/ml potassium sorbate, preferably about 1 mg/ml potassium sorbate.

The aqueous melatonin composition may also comprise a solvent enhancer, such as glycerol. Glycerol is odourless, colourless, and hygroscopic in nature due to the presence of three hydroxyl groups. Moreover, glycerol adds a sweet taste to liquid solutions.

Therefore, an embodiment of the present invention relates to the aqueous melatonin composition as described herein, wherein the aqueous phase comprises glycerol.

Another embodiment of the present invention relates to the aqueous melatonin composition as described herein, wherein the concentration of glycerol is in the range of about 70 mg/ml to 100 mg/ml, such as about 75 mg/ml to about 95 mg/ml, such as about 80 mg/ml to about 90 mg/ml, such as about 82 mg/ml to about 88 mg/ml, preferably about 85 mg/ml.

A further embodiment of the present invention relates to the aqueous melatonin composition as described herein, wherein said composition comprises;
i) about 0.5 mg/ml to about 2.0 mg/ml melatonin, preferably about 1 mg/ml melatonin,
ii) about 0.5 mg/ml to about 2.0 mg/ml potassium sorbate, preferably about 1 mg/ml potassium sorbate, and
iii) about 70 mg/ml to about 100 mg/ml glycerol, preferably about 85 mg/ml glycerol.

To optimise the stability of the aqueous melatonin composition it is preferred to adjust the pH of the composition. Without being bound by theory, it is contemplated that too high pH values may destabilise melatonin due to deprotonation of the amide functional group of the indole group in the melatonin structure. Accordingly, slightly acidic pH values are preferred. Adjustment of the pH can be done with any suitable acid, such as HCl, that does not interfere with the properties of the aqueous melatonin composition.

Therefore, an embodiment of the present invention relates to the aqueous melatonin composition as described herein, wherein the pH of said composition is in the range of about pH 3.5 to about pH 5.5, such as about pH 4.0 to about pH 5.0, such as about pH 4.2 to about pH 4.8, such as about pH 4.4 to about pH 4.6, preferably about pH 4.5.

As noted herein, the aqueous melatonin composition requires no additional preservative beyond potassium sorbate. Particularly, it is preferred to avoid some of the conventional preservatives that may cause adverse effects upon ingestion.

Thus, an embodiment of the present invention relates to the aqueous melatonin composition, wherein said additional preservatives are selected from the group consisting of parabens, benzoates, isothiazolinones, and antioxidants.

Another embodiment of the present invention relates to the aqueous melatonin composition, wherein said additional preservatives are selected from the group consisting of parabens, benzoates, isothiazolinones, antioxidants, and simple alcohols.

In the present context, the term “simple alcohols” refers to primary monoalcohols (RCH2OH), secondary monoalcohols (R2CHOH) and tertiary monoalcohols (R3COH).

It is to be understood that glycerol in the present context is not considered an additional preservative. Instead glycerol may be added to the composition as a solvent enhancer/thickening agent.

Thus, an embodiment of the present invention relates to the aqueous melatonin composition, wherein said additional preservatives does not include glycerol.

Another embodiment of the present invention relates to the aqueous melatonin composition as described herein, wherein said additional preservative are selected from the group consisting of parabens, sodium benzoate, benzyl alcohol, chloro-butanol, chloro-cresol, alkyl esters of paraben, phenol, phenyl ethanol, propylene glycol, chloroform, benzoic acid, and antioxidants.

It is also preferred to avoid inclusion of antioxidants in the aqueous melatonin composition. For example, citric acid, has been linked to side effects including muscle twitching or cramps, swelling or weight gain, weakness, mood changes, rapid and shallow breathing, fast heart rate, restless feeling, or diarrhoea. Accordingly, individuals suffering from kidney problems, heart problems or elevated blood pressure, adrenal gland disorders or hyperkalemia are not recommended to ingest superfluous citric acid. Moreover, it is well established that liquids comprising citric acid cause irreversible dental erosion (enamel erosion). Citric acid, especially synthetic, is further regarded as having strong negative influence on both individual and environmental health by active influence groups.

Accordingly, an embodiment of the present invention relates to the aqueous melatonin composition as described herein, wherein said composition is free of antioxidants.

Another embodiment of the present invention relates to the aqueous melatonin composition as described herein, wherein said antioxidants are selected from the group consisting of citric acid, lecithin, vitamin C, vitamin E, flavonoids, anthocyanins, genistein and quercetin.

A further embodiment of the present invention relates to the aqueous melatonin composition as described herein, wherein said antioxidant is citric acid.

An still further embodiment of the present invention relates to the aqueous melatonin composition as described herein, wherein said antioxidant is lecithin.

An even further embodiment of the present invention relates to the aqueous melatonin composition as described herein, wherein said antioxidant is citric acid and/or lecithin.

The intent with the melatonin composition described herein is to provide as mild a composition as possible for the benefit of the consumer. Liquid compositions are easy to administer to individuals that are not comfortable with swallowing any solid dosage forms, such as pills, and can easily be ingested orally. Thus, the composition is prepared as an aqueous composition mainly comprising water and very limited amounts of any other solvents. Particularly, organic solvents, such as alcohols, are preferably avoided.

Accordingly, an embodiment of the present invention relates to the aqueous melatonin composition as described herein, wherein said composition is a liquid composition.

Another embodiment of the present invention relates to the aqueous melatonin composition as described herein, wherein said composition is suitable for oral administration.

It is to be understood that the melatonin of the composition is fully dissolved in the aqueous phase. The appearance of the composition is that of a solution and not a suspension, dispersion or the like. This means that the melatonin is neither in a particulate form, such as micro- or nano-particulate form, nor in a aggregate system, such as lipid systems. Accordingly, unwanted aggregate systems may be liposomal or micellar systems comprising fatty acids and/or lipid compounds such as lecithin.

Thus, an embodiment of the present invention relates to the aqueous melatonin composition as described herein, wherein said composition is an aqueous solution.

Another embodiment of the present invention relates to the aqueous melatonin composition as described herein, wherein said composition does not comprise melatonin in particulate form, such as nanoparticulate form.

It is generally unwanted in medicinal products for human consumption to include organic solvents, such as alcohols. This is particularly true for products intended for consumption by children and/or adolescents.

Thus, a further embodiment of the present invention relates to the aqueous melatonin composition as described herein, wherein said composition is free of any alcohol other than glycerol.

Another embodiment of the present invention relates to the aqueous melatonin composition as described herein, wherein said composition is free of simple alcohols.

Yet another embodiment of the present invention relates to the aqueous melatonin composition as described herein, wherein said composition is free of ethanol.

A still further embodiment of the present invention relates to the aqueous melatonin composition as described herein, wherein said composition is free of xylitol.

The aqueous phase of the composition comprises both the active ingredient and the preservative. The aqueous phase is preferably comprising purified water to reduce the risk of any contaminants affecting the melatonin and the long-term stability and activity thereof. For some variants of the aqueous melatonin composition, the content of water (w/v) may be even higher than 85% w/v.

Thus, an embodiment of the present invention relates to the aqueous melatonin composition as described herein, wherein the aqueous phase comprises at least about 86% w/v water, such as about 87% w/v water, such as about 88% w/v water, such as about 89% w/v water, such as about 90% w/v water.

Another embodiment of the present invention relates to the aqueous melatonin composition as described herein, wherein said water is purified water.

In variants of the aqueous melatonin composition the aqueous phase is not supplemented with a thickening agent, but the viscoelastic properties are acceptable, optionally with the addition of glycerol.

Therefore, an embodiment of the present invention relates to the aqueous melatonin composition as described herein, wherein said composition is free of any thickening agents.

Another embodiment of the present invention relates to the aqueous melatonin composition as described herein, wherein said composition is free of any thickening agents other than glycerol.

A further embodiment of the present invention relates to the aqueous melatonin composition as described herein, wherein said composition is free of any polymeric thickening agents.

Polymeric thickening agents include, but is not limited to, a variety of gums and starches. Thus, an embodiment of the present invention relates to the aqueous melatonin composition as described herein, wherein said thickening agents are selected from the group consisting of xanthan gum, alginin, guar gum, locust bean gum, starches, carrageenan, and gelatin.

Yet another embodiment of the present invention relates to the aqueous melatonin composition as described herein, wherein said thickening agent is xanthan gum.

It may be desirable to add one or more flavouring agents to the composition to improve the organoleptic experience of the consumer. Such flavouring agents will only be added in small amounts that does not alter the physical or pharmacokinetic properties of the aqueous melatonin composition. The flavouring agents may be either natural (e.g. extracted from fruit, berry, stem or leaf) or synthetic (e.g. synthesized aroma chemicals).

Accordingly, an embodiment of the present invention relates to the aqueous melatonin composition as described herein, wherein said composition further comprises one or more flavouring agents.

Another embodiment of the present invention relates to the aqueous melatonin composition as described herein, wherein said flavouring agents have a taste selected from the group consisting of strawberry, mint, banana, apple, coconut, cherry, lemon, vanilla, pineapple, orange, and combinations thereof.

A further embodiment of the present invention relates to the aqueous melatonin composition as described herein, wherein the one or more flavouring agents are selected from the group consisting of maltol, vanillin, ethyl vanillin, menthol, malic acid, fumaric acid, ethyl maltol, tartaric acid, isoamyl acetate, gamma-octalactone, allyl hexanoate, ethyl butyrate, cinnamaldehyde, ethyl 2-methylpentanoate, ethyl methylphenyl glycidate and combinations thereof.

Certain aqueous melatonin compositions have been identified as favoured and with good stability upon storage and/or when challenged with microorganisms.

Therefore, an embodiment of the present invention relates to the aqueous melatonin composition as described herein, wherein said composition contains;
i) melatonin,
ii) potassium sorbate, and
iii) an aqueous solvent comprising purified water and glycerol,

Another embodiment of the present invention relates to the aqueous melatonin composition as described herein, wherein said composition contains;
i) melatonin,
ii) potassium sorbate,
iii) glycerol,
iv) hydrochloric acid, and
v) purified water.

A further embodiment of the present invention relates to the aqueous melatonin composition as described herein, wherein said composition contains;
i) about 1 mg/ml melatonin,
ii) about 1 mg/ml potassium sorbate,
iii) about 85 mg/ml glycerol,
iv) hydrochloric acid, and
v) purified water.

A still further embodiment of the present invention relates to the aqueous melatonin composition as described herein, wherein the pH of said composition is about pH 4.5.

Pharmaceutical compositions require some antimicrobial activity to prevent hazards to the consumer from infection and spoilage of the composition due to microbial proliferation during storage. This is particularly an issue with aqueous formulations. To this end, compounds with antimicrobial activity are added. For the aqueous melatonin composition described herein, the compound with antimicrobial activity is potassium sorbate. The storage stability of the aqueous melatonin composition may be determined by measurement of the antimicrobial activity in accordance with European Pharmacopoeia 10^{th} Edition, 5.1.3 Efficacy of antimicrobial preservation (Ph.Eur.5.1.3).

Hence, it is to be understood that storage stability in the present context relates to antimicrobial stability. Thus, storage stability does not refer to e.g. stabilisation of components of the composition or the ability of retaining (nano) particles in suspension.

Accordingly, an embodiment of the present invention relates to the aqueous melatonin composition as described herein, wherein storage stability is assessed according to Ph. Eur. 5.1.3 (version 10).

Another embodiment of the present invention relates to the aqueous melatonin composition according to any one of the preceding items, wherein the storage stability of the composition is at least 4 weeks as determined by measurement of the antimicrobial activity in accordance with European Pharmacopoeia 10^{th} Edition, 5.1.3 Efficacy of antimicrobial preservation (Ph.Eur.5.1.3).

A further embodiment of the present invention relates to the aqueous melatonin composition as described herein, wherein the composition is stable upon storage at room temperature for at least 12 months, such as at least 18 months, such as at least 24 months.

A still further embodiment of the present invention relates to the aqueous melatonin composition as described herein, wherein the composition has a storage stability at room temperature of at least 12 months, such as at least 18 months, such as at least 24 months.

The aqueous melatonin composition is a neurohormone that is known to play an active role in the circadian rhythm, and in particular affect the sleep-wake timing. Thus, it has found medical use for those individuals with non-regular sleeping patterns. As described herein, melatonin is preferred over conventional hypnotics because the risk of adverse effects and dependency is limited.

Thus, an aspect of the present invention relates to an aqueous melatonin composition as described herein for use as a medicament.

Another aspect of the present invention relates to an aqueous melatonin composition as described herein for use in treatment of sleep disorders.

The aqueous melatonin composition described herein is useful for treatment of sleep disorders, such as insomnia. Many variants of insomnia exist including, but not limited to, sleep maintenance insomnia, terminal insomnia, sleep onset insomnia, and psychophysiological insomnia. However, the aqueous melatonin composition may also be used in the treatment of disorder associated with the circadian rhythm including, but not limited to, jet lag, shift work sleep disorder, delayed sleep phase disorder (DSPS) and non-24-hour sleep wake disorder.

Therefore, an embodiment of the present invention relates to the aqueous melatonin composition as described herein, wherein the sleep disorders are selected from the group consisting of insomnia, sleep disorders associated with the circadian rhythm, delayed sleep phase disorder (DSPS), jet lag, sleep disorders associated with a psychiatric condition, sleep disorders associated with neurological disease, sleep disorders associated with a mental condition, sleep disorders associated with a medical disorder.

Another embodiment of the present invention relates to the aqueous melatonin composition as described herein, wherein the sleep disorder is a sleep disorders associated with the circadian rhythm.

Other patient groups that may benefit from the aqueous melatonin composition described herein are patients experiencing sleep disorders derived from psychiatric conditions and/or neurological disease. These patients may suffer from attention deficit hyperactivity disorder (ADHD), autism spectrum disorder (ASD), dementia or parkinsonism. Moreover, sleep disorders often occur in combination with mental disorders, such as psychoses or mood and anxiety disorders, or in conjunction with medical disorders, such as chronic obstructive pulmonary disease and nocturnal cardiac ischemia.

Accordingly, an embodiment of the present invention relates to the aqueous melatonin composition for use as described herein, wherein the sleep disorders is insomnia due to ADHD.

Another embodiment of the present invention relates to the aqueous melatonin composition for use as described herein, wherein the rapid release melatonin formulation is administered to an individual that has discontinued the use of a benzodiazepine or non-benzodiazepine hypnotic.

A further embodiment of the present invention relates to the aqueous melatonin composition for use as described herein, wherein the sleep disorder is insomnia.

The aqueous melatonin composition is formulated so it can suitably be administered by ingestion through the mouth. The recommended ingested dose may be varied depending on the subject to receive the treatment.

Thus, an embodiment of the present invention relates to the aqueous melatonin composition as described herein, wherein the composition is administered to a subject via the oral route.

Another embodiment of the present invention relates to the aqueous melatonin composition as described herein, wherein the composition is administered in a dose comprising 0.5-20 mg melatonin, such as 0.75-10 mg melatonin, such as 1-5 mg melatonin.

The subject to receive the treatment can in principle be any mammal, e.g. there could be situations where an animal, such as a pet animal, would benefit from administration of the aqueous melatonin composition. However, it is preferred that the subject is a human. Especially, children, adolescents and elderly that may be more vulnerable to adverse effects of excessive and potential harmful excipients may benefit from the aqueous melatonin composition described herein.

Therefore, an embodiment of the present invention relates to the aqueous melatonin composition as described herein, wherein the subject is a human.

Another embodiment of the present invention relates to the aqueous melatonin composition as described herein, wherein the subject is a child or adolescent.

A further embodiment of the present invention relates to aqueous melatonin composition as described herein, wherein the composition is administered to a child or adolescent with ADHD or a neuropsychiatric disorder.

A still further embodiment of the present invention relates to the aqueous melatonin composition for use as described herein, wherein the composition is administered to an adult aged 50 years or older, such as 55 years or older, such as 60 years or older, such as 70 years or older, such as 80 years or older.

The aqueous melatonin composition may be prepared in industrial scale by careful mixing of melatonin and potassium sorbate in an aqueous phase, followed by adjustment of pH and purification.

Accordingly, an aspect of the present invention relates to an aqueous melatonin composition as described herein obtained by a method as described herein.

Another aspect of the present invention relates to a method for preparing an aqueous melatonin composition as described herein, said method comprising the steps of:
i) Provision of an aqueous phase,
ii) Addition of potassium sorbate and melatonin to the aqueous phase to obtain an aqueous melatonin solution,
iii) Adjustment of the pH of said aqueous melatonin solution,
iv) Filtration of said aqueous melatonin solution, thereby obtaining said aqueous melatonin composition.

An embodiment of the present invention relates to the method as described herein, wherein said aqueous phase comprises purified water.

Glycerol may be added as a solvent enhancer to the aqueous phase of the composition. Therefore, an embodiment of the present invention relates to the method as described herein, wherein said aqueous phase comprises glycerol.

It has been found that distribution of the ingredient of the composition can be effectively achieved by continuous stirring, e.g. with a magnetic stirrer, a propeller, preferably a propeller. The production time can be reduced by selecting the velocity of stirring to efficiently dissolve the ingredients without splashing or foaming.

Therefore, an embodiment of the present invention relates to the method as described herein, wherein the addition of potassium sorbate and melatonin is performed under continuous stirring.

Another embodiment of the present invention relates to the method as described herein, wherein said stirring is performed in three consecutive steps of firstly about 575 rpm, secondly about 350 rpm, and thirdly about 200 rpm.

The pH of the aqueous melatonin composition can be adjusted by any suitable acid that does not inadvertently alter the properties of the composition. This acid may be HCl. The acid may conveniently be titrated into the composition until the desired pH is achieved.

Thus, an embodiment of the present invention relates to the method as described herein, wherein adjustment of the pH is performed using hydrochloric acid (HCl).

Another embodiment of the present invention relates to the method as described herein, wherein the pH is adjusted to a pH in the range of about 4.4 to about 4.6, preferably about 4.5.

The aqueous melatonin composition is filtered to remove any particulate matters. Filtering of the solution can be achieved by passing the composition through membrane filters of a defined pore size.

Therefore, an embodiment of the present invention relates to the method as described herein, wherein filtration is performed with membrane filters with a pore size in the range of about 0.5 μm to about 1.5 μm, such as about 0.8 μm to about 1.2 μm, preferably about 1.0 μm.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the aqueous melatonin composition and all its features, which may readily be part of the method as described herein.

Embodiments and features of the present invention are also outlined in the following items.

### Items

X1. An aqueous melatonin composition comprising:
i) an active ingredient selected from melatonin, and
ii) a preservative selected from potassium sorbate, dissolved in an aqueous phase,
   wherein the aqueous phase comprises at least about 85% w/v water, and
   wherein the composition is free of any additional preservatives.

X2. The aqueous melatonin composition according to item X1, wherein the melatonin is in the form of micronized melatonin.

X3. The aqueous melatonin composition according to any one of items X1 or X2, wherein the size of the micronized melatonin (D90) is in the range of about 5 μm to about 20 μm, such as about 5 μm to about 10 μm.

X4. The aqueous melatonin composition according to any one of the preceding items, wherein the concentration of melatonin is in the range of about 0.5 mg/ml to about 2.0 mg/ml, such as about 0.75 mg/ml to about 1.5 mg/ml, such as about 0.9 mg/ml to about 1.1 mg/ml, preferably about 1 mg/ml.

X5. The aqueous melatonin composition according to any one of the preceding items, wherein said composition is free of additional active ingredients.

X6. The aqueous melatonin composition according to any one of the preceding items, wherein the concentration of potassium sorbate is less than about 2.0 mg/ml, such as less than about 1.5 mg/ml, such as less than about 1.2 mg/ml.

X7. The aqueous melatonin composition according to any one of the preceding items, wherein the concentration of potassium sorbate is in the range of about 0.5 mg/ml to about 2.0 mg/ml, such as about 0.75 mg/ml to about 1.5 mg/ml, such as about 0.9 mg/ml to about 1.1 mg/ml, preferably about 1 mg/ml.

X8. The aqueous melatonin composition according to any one of the preceding items, wherein said composition comprises;
i) about 0.5 mg/ml to about 2.0 mg/ml melatonin, preferably about 1 mg/ml melatonin, and
ii) about 0.5 mg/ml to about 2.0 mg/ml potassium sorbate, preferably about 1 mg/ml potassium sorbate.

X9. The aqueous melatonin composition according to any one of the preceding items, wherein the aqueous phase comprises glycerol.

X10. The aqueous melatonin composition according to item X9, wherein the concentration of glycerol is in the range of about 70 mg/ml to 100 mg/ml, such as about 75 mg/ml to about 95 mg/ml, such as about 80 mg/ml to about 90 mg/ml, such as about 82 mg/ml to about 88 mg/ml, preferably about 85 mg/ml.

X11. The aqueous melatonin composition according to any one of items X9 or X10, wherein said composition comprises;
i) about 0.5 mg/ml to about 2.0 mg/ml melatonin, preferably about 1 mg/ml melatonin,
ii) about 0.5 mg/ml to about 2.0 mg/ml potassium sorbate, preferably about 1 mg/ml potassium sorbate, and
iii) about 70 mg/ml to about 100 mg/ml glycerol, preferably about 85 mg/ml glycerol.

X12. The aqueous melatonin composition according to any one of the preceding items, wherein the pH of said composition is in the range of about pH 3.5 to about pH 5.5, such as about pH 4.0 to about pH 5.0, such as about pH 4.2 to about pH 4.8, such as about pH 4.4 to about pH 4.6, preferably about pH 4.5.

X13. The aqueous melatonin composition according to any one of the preceding items, wherein said additional preservative are selected from the group consisting of parabens, sodium benzoate, benzyl alcohol, chloro-butanol, chloro-cresol, alkyl esters of paraben, phenol, phenyl ethanol, propylene glycol, chloroform, benzoic acid, and antioxidants.

X14. The aqueous melatonin composition according to any one of the preceding items, wherein said composition is free of antioxidants.

X15. The aqueous melatonin composition according to any one of items X13 or X14, wherein said antioxidants are selected from the group consisting of citric acid, lecithin, vitamin C, vitamin E, flavonoids, anthocyanins, genistein and quercetin.

X16. The aqueous melatonin composition according to any one of items X13-X15, wherein said antioxidant is citric acid.

X17. The aqueous melatonin composition according to any one of the preceding items, wherein said composition is free of any alcohol other than glycerol.

X18. The aqueous melatonin composition according to any one of the preceding items, wherein said composition is free of xylitol.

X19. The aqueous melatonin composition according to any one of the preceding items, wherein said composition is free of any thickening agents.

X20. The aqueous melatonin composition according to item X19, wherein said thickening agents are selected from the group consisting of xanthan gum, alginin, guar gum, locust bean gum, starches, carrageenan, and gelatin.

X21. The aqueous melatonin composition according to any one of items X19 or X20, wherein said thickening agent is xanthan gum.

X22. The aqueous melatonin composition according to any one of the preceding items, wherein said composition is a liquid composition.

X23. The aqueous melatonin composition according to any one of the preceding items, wherein said composition is suitable for oral administration.

X24. The aqueous melatonin composition according to any one of the preceding items, wherein the aqueous phase comprises at least about 86% w/v water, such as about 87% w/v water, such as about 88% w/v water, such as about 89% w/v water, such as about 90% w/v water.

X25. The aqueous melatonin composition according to any one of the preceding items, wherein said water is purified water.

X26. The aqueous melatonin composition according to any one of the preceding items, wherein said composition further comprises one or more flavouring agents.

X27. The aqueous melatonin composition according to item X26, wherein said flavouring agents have a taste selected from the group consisting of strawberry, mint, banana, apple, coconut, cherry, lemon, vanilla, pineapple, orange, and combinations thereof.

X28. The aqueous melatonin composition according to any one of the preceding items, wherein said composition contains;
i) melatonin,
ii) potassium sorbate, and
iii) an aqueous solvent comprising purified water and glycerol,

X29. The aqueous melatonin composition according to any one of the preceding items, wherein said composition contains;
i) melatonin,
ii) potassium sorbate,
iii) glycerol,
iv) hydrochloric acid, and
v) purified water.

X30. The aqueous melatonin composition according to any one of the preceding items, wherein said composition contains;
i) about 1 mg/ml melatonin,
ii) about 1 mg/ml potassium sorbate,
iii) about 85 mg/ml glycerol,
iv) hydrochloric acid, and
v) purified water.

X31. The aqueous melatonin composition according to any one of the preceding items, wherein the pH of said composition is about pH 4.5.

X32. The aqueous melatonin composition according to any one of the preceding items, wherein the composition is stable upon storage at room temperature for at least 12 months, such as at least 18 months, such as at least 24 months.

X33. The aqueous melatonin composition according to item 32, wherein storage stability is assessed according to Ph. Eur. 5.1.3 (version 10).

Y1. An aqueous melatonin composition according to any one of the preceding items for use as a medicament.

Y2. An aqueous melatonin composition according to any one of the preceding items for use in treatment of sleep disorders.

Y3. The aqueous melatonin composition for use according to item Y2, wherein the sleep disorders are selected from the group consisting of insomnia, sleep disorders associated with the circadian rhythm, delayed sleep phase disorder (DSPS), jet lag, sleep disorders associated with a psychiatric condition, sleep disorders associated with neurological disease, sleep disorders associated with a mental condition, sleep disorders associated with a medical disorder.

Y4. The aqueous melatonin composition for use according to any one of items Y1-Y3, wherein the composition is administered to a subject via the oral route.

Y5. The aqueous melatonin composition for use according to any one of items Y1-Y4, wherein the composition is administered in a dose comprising 0.5-20 mg melatonin, such as 0.75-10 mg melatonin, such as 1-5 mg melatonin.

Y6. The aqueous melatonin composition for use according to any one of items Y1-Y5, wherein the subject is a human.

Y7. The aqueous melatonin composition for use according to any one of items Y1-Y6, wherein the subject is a child or adolescent.

W1. A method for preparing an aqueous melatonin composition according to any one of items X1-X33, said method comprising the steps of:
i) Provision of an aqueous phase,
ii) Addition of potassium sorbate and melatonin to the aqueous phase to obtain an aqueous melatonin solution,
iii) Adjustment of the pH of said aqueous melatonin solution,
iv) Filtration of said aqueous melatonin solution, thereby obtaining said aqueous melatonin composition.

W2. The method according to item W1, wherein said aqueous phase comprises purified water.

W3. The method according to any one of items W1 or W2, wherein said aqueous phase comprises glycerol.

W4. The method according to any one of items W1-W3, wherein the addition of potassium sorbate and melatonin is performed under continuous stirring.

W5. The method according to item W4, wherein said stirring is performed in three consecutive steps of firstly about 575 rpm, secondly about 350 rpm, and thirdly about 200 rpm.

W6. The method according to any one of items W1-W5, wherein adjustment of the pH is performed using hydrochloric acid (HCl).

W7. The method according to any one of items W1-W6, wherein the pH is adjusted to a pH in the range of about 4.4 to about 4.6, preferably about 4.5.

W8. The method according to any one of items W1-W7, wherein filtration is performed with membrane filters with a pore size in the range of about 0.5 μm to about 1.5 μm, such as about 0.8 μm to about 1.2 μm, preferably about 1.0 μm.

Z1. An aqueous melatonin composition according to any one of items X1-X33 obtainable by a method according to any one of items W1-W8.

The invention will now be described in further details in the following non-limiting examples.

### EXAMPLES

### Example 1: Preparation of Aqueous Melatonin Composition

This example describes how an exemplary version of the aqueous melatonin composition may be prepared. The exemplary composition was according to Table 1.

TABLE 1
Exemplary composition.

| | | Amount | Amount |
|---|---|---|---|
| Ingredient | Supplier | (mg/ml) | (% w/v) |
| Micronized melatonin | Flamma | 1.0 | 0.1 |
| Potassium sorbate | Sigma-Aldrich | 1.0 | 0.1 |
| Glycerol | Sigma-Aldrich | 85 | 8.5 |
| Hydrochloric acid (2M) | Sigma-Aldrich | q.s to pH 4.5 | q.s to pH 4.5 |
| Purified water | Solvecco | to 1000 | to 100 |

A batch of 80 litres of 1 mg/mL melatonin solution with a final weight of 81.6 kg were prepared using the raw materials according to Table 2.

TABLE 2
Amounts utilized for exemplary composition.

| Ingredient | Amount (g) |
|---|---|
| Micronized melatonin | 80 |
| Potassium sorbate | 80 |
| Glycerol | 6800 |
| Hydrochloric acid (2M) to pH 4.5 | Approx. 160 |
| Purified water | to 81600* |

*Corresponding volume is 80 L (density 1.02 g/mL).

### Method

The manufacturing process consists of three steps, wherein the final step is only necessary for storage and distribution:
Step 1: Preparation of the oral solution
Step 2: Filtration
Step 3: Filling of the bulk solution into the primary packaging

Step 1: preparation of the oral solution A 100 L stainless-steel vessel was used for preparation of the oral solution.

The ingredients were dispensed and added to the stainless-steel vessel in the following order:
1. The main part of purified water was charged to the mixing vessel and then glycerol was added. Glycerol was dissolved by mixing with a propeller overhead stirrer.
2. Potassium sorbate and micronized melatonin were added to the glycerol in water solution and dissolved under continuous mixing using the propeller overhead stirrer at as high speed as possible without splashing (˜575 rpm). When almost all solid ingredients were dissolved, the mixing speed was decreased to reduce the amount of foam (˜350 rpm). Stirring was continued at a slower speed (˜200 rpm) until a clear solution without foam was obtained. Dissolution of melatonin and potassium sorbate was checked and verified by visual inspection.
3. pH of the oral solution in the preparation vessel was measured and hydrochloric acid (2M) was added slowly to the solution under continuous mixing at low speed to obtain a target pH of 4.5 (measured with Mettler Toledo Seven2Go).
4. The final weight of the formulation was adjusted by addition of the remaining amount of purified water. Finally, pH was verified to be 4.5.

### Step 2: Filtration

The finished oral solution was filtered to remove any particulate matters using a disposable membrane filter (Pall Nova Star, pore size of 1.0 μm). The membrane filter was assembled with silicone tubes of 8 mm inner diameter (Watson-Marlow, Flexicon Accusil) and the solution was pumped through the membrane filter into stainless-steel vessels with lids using a peristaltic pump (Flexicon PF7).

Step 3: Filling into Packaging

The filtered oral solution was filled into 100 mL amber glass bottles using a Flexicon PF7 filling machine through silicone tubes of 8 mm inner diameter (Watson-Marlow, Flexicon Accusil). The filled bottles were sealed immediately with polypropylene (PP) snap-on caps.

### Results

The process produced clear aqueous melatonin compositions wherein in all ingredients were properly mixed and dissolved. pH was controlled to obtain maximum stability of the composition.

### Conclusion

A protocol to reliably and reproducibly produce aqueous melatonin compositions were established. This protocol was utilised to prepare a variety of melatonin and placebo compositions to evaluate the characteristics of the aqueous melatonin composition (example 2-4).

### Example 2: Testing Organoleptic Properties of Aqueous Composition

This example sets out to test how the ingredients of the aqueous melatonin composition influence the organoleptic properties of the formulation. Appearance and odour were the organoleptic properties evaluated.

### Method

Compositions were prepared according to the protocol described in example 1, with the exception that either potassium sorbate and/or methyl paraben was added as preservative(s) according to Table 3. For placebo samples without melatonin, the addition of melatonin was omitted.

The batch sizes were smaller than in example 1; 2000 mL (sample A), 600 mL (samples B and C), and 700 mL (sample D).

TABLE 3
Compositions for test of organoleptic properties.

| | Sample A | Sample B | Sample C | Sample D |
|---|---|---|---|---|
| Ingredient | (mg/ml) | (mg/ml) | (mg/ml) | (mg/ml) |
| Micronized melatonin | — | — | — | 1.0 |
| Potassium sorbate | 1.0 | — | 1.0 | 1.0 |
| Methyl paraben | 1.0 | 1.0 | — | 1.0 |
| Glycerol | 85 | 85 | 85 | 85 |
| Hydrochloric acid (2M) | to pH | to pH | to pH | to pH |
| | 4.4-4.6 | 4.4-4.6 | 4.4-4.6 | 4.4-4.6 |
| Purified water | to 1 | to 1 | to 1 | to 1 |

An accelerated stability study at 40° C./75% RH was performed to investigate the organoleptic properties of the compositions over time. All studies were repeated in replicas on 6, i.e. storage of 6 separate bottles with sample A-D.

### Results

The results of the development of appearance and odour of the compositions are presented in Table 4. The composition comprising the active ingredient (sample D) showed a deeper yellow colour than the placebo compositions (samples A-C). The omission of a second preservative did not cause a change of the colour of the composition.

Importantly, the medicine odour identified in the compositions comprising methyl paraben was not present in the composition comprising only potassium sorbate. Thus, the odour was caused by methyl paraben.

TABLE 4
Organoleptic properties of compositions over time.

| | | Time | | |
|---|---|---|---|---|
| Sample | Attribute | 0 | 1 month | 6 months |
| A | Appearance | Slightly more yellow than | Slightly more yellow than | Slightly more yellow than |
| | | water | water | water |
| | Odour | Slight smell of medicine | Slight smell of medicine | Smells like medicine |
| B | Appearance | Slightly more yellow than | Slightly more yellow than | Slightly more yellow than |
| | | water | water | water |
| | Odour | Slight smell of medicine | Slight smell of medicine | Smells like medicine |
| C | Appearance | Slightly more yellow than | Slightly more yellow than | Slightly more yellow than |
| | | water | water | water |
| | Odour | No smell | No smell | Faint clinical odour |
| D | Appearance | Slightly more yellow than | Slightly yellow solution | Clearly yellow |
| | | water | | |
| | Odour | Slight smell of medicine | Slight smell of medicine | Faintly like medicine |

### Conclusion

This example demonstrates that potassium sorbate does not cause unpleasant odour of the composition and does not negatively contribute to the colouring of the composition.

### Example 3: Testing Microbial Stability of Aqueous Melatonin Composition

This example evaluates the efficacy of antimicrobial preservation of aqueous melatonin compositions with different preservatory means.

### Method

Compositions were prepared according to the protocol described in example 1, with the exception that either potassium sorbate and/or methyl paraben was added as preservative(s) according to Table 5. For BN838 no preservative was added.

TABLE 5
Compositions for PET study.

| | Sample | Sample | Sample | Sample |
|---|---|---|---|---|
| | BL716 | BN836 | BN837 | BN838 |
| Ingredient | (mg/ml) | (mg/ml) | (mg/ml) | (mg/ml) |
| Micronized melatonin | 1.0 | 1.0 | 1.0 | 1.0 |
| Potassium sorbate | 1.0 | 0.5 | 1.0 | — |
| Methyl paraben | 1.0 | 0.5 | — | — |
| Glycerol | 85 | 85 | 85 | 85 |
| Hydrochloric acid (2M) | to pH 4.5 | to pH 4.5 | to pH 4.5 | to pH 4.5 |
| Purified water | to 1 | to 1 | to 1 | to 1 |

A modified preservative efficacy test (PET) was performed using Aspergillus brasiliensis (ATCC 16404), Candida albicans (ATCC 10231), Escherichia coli (ATCC 8739), Pseudomonas aeruginosa (ATCC 9027) and Staphylococcus aureus (ATCC 6538). The methodology was based on USP <51> and Ph. Eur. 5.1.3 for oral preparations, and the results was evaluated against Ph. Eur. 5.1.3 utilizing the acceptance criteria described therein.

### Media

The following media were used; 0.9% NaCl, 0.9% NaCl+0.05% Tween80, buffered NaCl peptone (BNP) with 1% Tween80, sabouraud dextrose agar (SDA), and tryptone soy agar (TSA).

### Microorganisms

Frozen stock suspensions of the microorganisms were used. The microorganisms were not more than 5 passages from the original master seed-lot. Microorganisms were thawed and mixed before use. Microorganisms were inoculated on solid agar media. After incubation, the microorganisms were harvested with 0.9% NaCl (0.9% NaCl with 0.05% Tween 80 for A. brasiliensis) and the concentration of the suspensions was adjusted to about 1×10⁸ colony forming units (cfu)/mL. Media and incubation times were according to Ph Eur 5.1.3.

### Assessment of Method Suitability/Validation Study

A method suitability assessment was performed and approved in advance to verify the suitability of the filtration method used in the test. 1 ml of sample BL716 was mixed with 100 ml of BNP with 1% Tween 80. 10 mL was added to filter funnels in duplicate, inoculated with a test organism and then filtered. The filters were rinsed with 3×100 mL of 0.9% NaCl before they were placed on TSA or SDA plates and incubated. For reference, 10 mL of BNP with 1% Tween 80 was inoculated and filtered in the same manner. The recovery of the inoculum, i.e. the ratio between growth in the inoculated product and the reference, should be between 50 and 200% for all microorganisms.

### Test for Efficacy of Antimicrobial Preservation

The test for efficacy of antimicrobial preservation was performed on 15 ml of the samples which were mixed in sterile containers with about 10⁴-10⁵ cfu/ml of each microorganism, separately. As reference, representing day 0, an equal concentration of all microorganisms was added to one bottle each containing 20 mL of 0.9% NaCl. The references were 10-fold serial diluted and tested immediately in duplicates by the surface spread method.

The inoculated sample was incubated at 20-25° C. for 28 days and tested at day 14 and day 28. 1 mL of the sample was mixed in 100 mL of BNP with 1% Tween 80. Different amounts (0.1, 1, and 10 mL) of the solution were filtered, after which the filters were rinsed three times with 100 ml of 0.9% NaCl. The filters were then placed on Tryptone Soy Agar (TSA) plates for bacteria and Sabouraud Dextrose Agar (SDA) plates for fungi.

E. coli, P. aeruginosa and S. aureus were incubated at 30-35° C. for more than 72 hours. C. albicans was incubated at 20-25° C. for more than 72 hours. A. brasiliensis was incubated at 20-25° C. for more than 120 hours. After incubation, the number of cfu was counted on each plate.

### Results

Data from the validation study are presented in Table 6 and show that all microorganisms grew within the specified limits.

TABLE 6
Viable count of inocula and the recovery relative
to the positive reference for each microorganism.

| | Viable count | Viable count | Recovery, |
|---|---|---|---|
| | sample | reference | Sample/reference |
| Microorganism | (cfu/plate) | (cfu/plate) | (%) |
| Aspergillus brasiliensis | 24, 32 | 24, 33 | 98 |
| Candida albicans | 37, 50 | 40, 45 | 102 |
| Escherichia coli | 48, 54 | 46, 49 | 107 |
| Pseudomonas aeruginosa | 29, 31 | 30, 34 | 94 |
| Staphylococcus aureus | 30, 37 | 38, 40 | 86 |

The results from the PET study are presented in Tables 7 and 8 and a comparison to the acceptance criteria stated in Ph Eur 5.1.3 in Table 9.

TABLE 7
Viable counts of each fungus in the presence of the sample,
presented as cfu/dilution and log₁₀ values (log₁₀ to the
mean value of the cfu/dilution). Day 0 corresponds to
the viable count of the reference, i.e. initial count.

| Sample | Sampling | A. brasiliensis | | C. albicans | |
|---|---|---|---|---|---|
| no | time | cfu/mL | log₁₀ | cfu/mL | log₁₀ |
| BL716 | Day 0 | 28 × 10⁴ | 5.45 | 51.5 × 10⁴ | 5.71 |
| | Day 14 | 2 × 10¹ | 1.3 | 0 × 10¹ | <1.00 |
| | Day 28 | 0 × 10¹ | <1.00 | 0 × 10¹ | <1.00 |
| B836 | Day 0 | 19.5 × 10⁴ | 5.29 | 79 × 10⁴ | 5.90 |
| | Day 14 | 14 × 10³ | 4.15 | 125 × 10¹ | 3.10 |
| | Day 28 | 7 × 10³ | 3.85 | 0 × 10¹ | <1.00 |
| B837 | Day 0 | 19.5 × 10⁴ | 5.29 | 79 × 10⁴ | 5.90 |
| | Day 14 | 0 × 10¹ | <1.00 | 12 × 10¹ | 2.08 |
| | Day 28 | 1 × 10¹ | 1.00 | 0 × 10¹ | <1.00 |
| B838 | Day 0 | 19.5 × 10⁴ | 5.29 | 79 × 10⁴ | 5.90 |
| | Day 14 | TNTC | >5.70 | 596 × 10³ | 5.78 |
| | Day 28 | 61 × 10⁴ | 5.79 | 47 × 10⁴ | 5.67 |

TABLE 8
Viable counts of each bacterium in the presence of the sample, presented as
cfu/dilution and log₁₀ values (log₁₀ to the mean value of the cfu/dilution).
Day 0 corresponds to the viable count of the reference, i.e. initial count.

| Sample | Sampling | E. coli | | P. aeruginosa | | S. aureus | |
|---|---|---|---|---|---|---|---|
| no | time | cfu/mL | log₁₀ | cfu/mL | log₁₀ | cfu/mL | log₁₀ |
| BL716 | Day 0 | 81.5 × 10⁴ | 5.91 | 33 × 10⁴ | 5.52 | 18.5 × 10⁴ | 5.27 |
| | Day 14 | 0 × 10¹ | <1.00 | 0 × 10¹ | <1.00 | 0 × 10¹ | <1.00 |
| | Day 28 | 0 × 10¹ | <1.00 | 0 × 10¹ | <1.00 | 0 × 10¹ | <1.00 |
| B836 | Day 0 | 95 × 10⁴ | 5.98 | 29.5 × 10⁴ | 5.47 | 46 × 10⁴ | 5.66 |
| | Day 14 | 0 × 10¹ | <1.00 | 0 × 10¹ | <1.00 | 0 × 10¹ | <1.00 |
| | Day 28 | 0 × 10¹ | <1.00 | 0 × 10¹ | <1.00 | 0 × 10¹ | <1.00 |
| B837 | Day 0 | 95 × 10⁴ | 5.98 | 29.5 × 10⁴ | 5.47 | 46 × 10⁴ | 5.66 |
| | Day 14 | 0 × 10¹ | <1.00 | 0 × 10¹ | <1.00 | 0 × 10¹ | <1.00 |
| | Day 28 | 0 × 10¹ | <1.00 | 0 × 10¹ | <1.00 | 0 × 10¹ | <1.00 |
| B838 | Day 0 | 95 × 10⁴ | 5.98 | 29.5 × 10⁴ | 5.47 | 46 × 10⁴ | 5.66 |
| | Day 14 | 10 × 10³ | 4.00 | 0 × 10¹ | <1.00 | 0 × 10¹ | <1.00 |
| | Day 28 | 20 × 10³ | 4.30 | 0 × 10¹ | <1.00 | 0 × 10¹ | <1.00 |

TABLE 9
Criteria and results of PET study in fungi and bacteria.
Results are presented as log₁₀ differences between day
0, day 14 and day 28, respectively. Day 0 corresponds to
the viable count of the reference, i.e. initial count.

| Sample | | Comparison | Acceptance | Log₁₀ | |
|---|---|---|---|---|---|
| no | Microbial | time | criteria | reduction | Result |
| BL716 | Bacteria | Day 14 | 3 | >4.3 | >4 |
| | | Day 28 | NI | >4.3 | NI |
| | Fungi | Day 14 | 1 | ≥4.2 | ≥4 |
| | | Day 28 | NI | >4.5 | NI |
| B836 | Bacteria | Day 14 | 3 | >4.5 | >4 |
| | | Day 28 | NI | >4.5 | NI |
| | Fungi | Day 14 | 1 | ≥1.1 | ≥1 |
| | | Day 28 | NI | ≥1.4 | NI |
| B837 | Bacteria | Day 14 | 3 | >4.5 | >4 |
| | | Day 28 | NI | >4.5 | NI |
| | Fungi | Day 14 | 1 | ≥3.8 | ≥4 |
| | | Day 28 | NI | ≥4.3 | NI |
| B838 | Bacteria | Day 14 | 3 | ≥2.0 | ≥2 |
| | | Day 28 | NI | ≥1.7 | NI |
| | Fungi | Day 14 | 1 | — | 1 |
| | | Day 28 | NI | — | 1 |

NI = No increase in number of viable microorganisms compared to the previous reading.

All samples comprising preservatives (BL716, BN836 and BN837) met the log 10 reduction criteria stated in Ph. Eur. 5.1.3 for both bacteria and fungi. The control sample without preservative (BN838) did not fulfil the criteria of Ph. Eur. 5.1.3.

Notably, the sample comprising only potassium sorbate (1 mg/mL, BN837) performed equally well overall as the sample comprising both potassium sorbate and methyl paraben in the double amount (2 mg/mL, BL716) and markedly better against fungi than the sample comprising both potassium sorbate and methyl paraben in the same amount (1 mg/mL, BN836).

### Conclusion

This example demonstrates that efficient antimicrobial preservation of aqueous melatonin compositions can be achieved by using only potassium sorbate as preservative.

### Example 4: Testing Boundaries of Aqueous Melatonin Composition

This example probed the limits of potassium sorbate and pH of the aqueous melatonin composition through a PET study.

### Method

Compositions were prepared according to the protocol described in example 1. Three compositions were prepared according to Table 9.

TABLE 10
Compositions for PET study.

| | Sample | Sample | Sample |
|---|---|---|---|
| | BS801 | BS802 | BS803 |
| Ingredient | (mg/ml) | (mg/ml) | (mg/ml) |
| Micronized melatonin | 1.0 | 1.0 | 1.0 |
| Potassium sorbate | 1.0 | 0.9 | 0.9 |
| Glycerol | 85 | 85 | 85 |
| Hydrochloric acid (2M) | to pH 4.5 | to pH 4.5 | to pH 4.7 |
| Purified water | to 1 | to 1 | to 1 |

The media, microorganisms and methodology of this example was identical to that of Example 3.

### Results

The results from the PET study are presented in Tables 11 and 12 and a comparison to the acceptance criteria stated in Ph Eur 5.1.3 in Table 13.

TABLE 11
Viable counts of each fungus in the presence of the sample,
presented as cfu/dilution and log₁₀ values (log₁₀ to the
mean value of the cfu/dilution). Day 0 corresponds to
the viable count of the reference, i.e. initial count.

| Sample | Sampling | A. brasiliensis | | C. albicans | |
|---|---|---|---|---|---|
| no | time | cfu/mL | log₁₀ | cfu/mL | log₁₀ |
| BS801 | Day 0 | 12.5 × 10⁴ | 5.1 | 76 × 10⁵ | 6.9 |
| | Day 14 | 16 × 10¹ | 2.2 | 42 × 10¹ | 2.6 |
| | Day 28 | 8 × 10¹ | 1.9 | 0 × 10¹ | <1.00 |
| BS802 | Day 0 | 12.5 × 10⁴ | 5.1 | 76 × 10⁵ | 6.9 |
| | Day 14 | 14.5 × 10¹ | 2.2 | 37 × 10³ | 4.6 |
| | Day 28 | 6 × 10¹ | 1.8 | 0 × 10¹ | <1.00 |
| BS803 | Day 0 | 12.5 × 10⁴ | 5.1 | 76 × 10⁵ | 6.9 |
| | Day 14 | 4.5 × 10³ | 3.7 | 152 × 10³ | 5.2 |
| | Day 28 | 3.5 × 10³ | 3.5 | 0 × 10¹ | <1.00 |

TABLE 12
Viable counts of each bacterium in the presence of the sample, presented as
cfu/dilution and log₁₀ values (log₁₀ to the mean value of the cfu/dilution).
Day 0 corresponds to the viable count of the reference, i.e. initial count.

| Sample | Sampling | E. coli | | P. aeruginosa | | S. aureus | |
|---|---|---|---|---|---|---|---|
| no | time | cfu/mL | log₁₀ | cfu/mL | log₁₀ | cfu/mL | log₁₀ |
| BS801 | Day 0 | 50.5 × 10⁴ | 5.7 | 49 × 10⁴ | 5.7 | 90 × 10⁴ | 6.0 |
| | Day 14 | 0 × 10¹ | <1.00 | 0 × 10¹ | <1.00 | 0 × 10¹ | <1.00 |
| | Day 28 | 0 × 10¹ | <1.00 | 0 × 10¹ | <1.00 | 0 × 10¹ | <1.00 |
| BS802 | Day 0 | 50.5 × 10⁴ | 5.7 | 49 × 10⁴ | 5.7 | 90 × 10⁴ | 6.0 |
| | Day 14 | 0 × 10¹ | <1.00 | 0 × 10¹ | <1.00 | 0 × 10¹ | <1.00 |
| | Day 28 | 0 × 10¹ | <1.00 | 0 × 10¹ | <1.00 | 0 × 10¹ | <1.00 |
| BS803 | Day 0 | 50.5 × 10⁴ | 5.7 | 49 × 10⁴ | 5.7 | 90 × 10⁴ | 6.0 |
| | Day 14 | 0 × 10¹ | <1.00 | 0 × 10¹ | <1.00 | 0 × 10¹ | <1.00 |
| | Day 28 | 0 × 10¹ | <1.00 | 0 × 10¹ | <1.00 | 0 × 10¹ | <1.00 |

TABLE 13
Criteria and results of PET study in fungi and bacteria. Results
are presented as log₁₀ differences between day 0, day 14 and
day 28, respectively. Day 0 corresponds to the viable count of
the reference, i.e. initial count NI = No increase in number
of viable microorganisms compared to the previous reading.

| Sample | | Comparison | Acceptance | Log₁₀ | |
|---|---|---|---|---|---|
| no | Microbial | time | criteria | reduction | Result |
| BS801 | Bacteria | Day 14 | 3 | >4.5 | >4 |
| | | Day 28 | NI | >4.5 | NI |
| | Fungi | Day 14 | 1 | ≥2.9 | >2 |
| | | Day 28 | NI | ≥3.2 | NI |
| BS802 | Bacteria | Day 14 | 3 | >4.5 | >4 |
| | | Day 28 | NI | >4.5 | NI |
| | Fungi | Day 14 | 1 | ≥2.3 | >2 |
| | | Day 28 | NI | ≥3.3 | NI |
| BS803 | Bacteria | Day 14 | 3 | >4.5 | >4 |
| | | Day 28 | NI | >4.5 | NI |
| | Fungi | Day 14 | 1 | ≥1.4 | ≥1 |
| | | Day 28 | NI | ≥1.6 | NI |

All samples met the log₁₀ reduction criteria stated in Ph. Eur. 5.1.3 for both bacteria and fungi. No significant difference was observed between samples comprising 1 mg/ml (BS801) and 0.9 mg/ml (BS802) potassium sorbate. The preservatory effect of potassium sorbate was decreased against fungi at pH 4.7 (BS803).

### Conclusion

This example demonstrates that potassium sorbate provides efficient antimicrobial preservation of aqueous melatonin compositions also when the concentration is lowered slightly, or the pH is varied.

1. An aqueous melatonin composition comprising:
melatonin, and
potassium sorbate,
dissolved in an aqueous phase,
wherein the aqueous phase comprises at least about 85% w/v water, and
wherein the composition is free of any additional preservatives.

2. The aqueous melatonin composition according to claim 1, wherein the concentration of melatonin is in the range of about 0.5 mg/ml to about 2.0 mg/ml.

3. The aqueous melatonin composition according to claim 1, wherein said composition is free of additional active ingredients.

4. The aqueous melatonin composition according to claim 1, wherein the concentration of potassium sorbate is in the range of about 0.5 mg/ml to about 2.0 mg/ml.

5. The aqueous melatonin composition according to claim 1, wherein the aqueous phase comprises glycerol.

6. The aqueous melatonin composition according to claim 1, wherein said additional preservative are selected from the group consisting of parabens, sodium benzoate, benzyl alcohol, chloro-butanol, chloro-cresol, alkyl esters of paraben, phenol, phenyl ethanol, propylene glycol, chloroform, benzoic acid, and antioxidants.

7. The aqueous melatonin composition according to claim 1, wherein said composition is free of any alcohol other than glycerol.

8. The aqueous melatonin composition according to claim 1, wherein said composition is free of any thickening agents.

9. The aqueous melatonin composition according to claim 1, wherein the composition is stable upon storage at room temperature for at least 12 months.

10. A medicament comprising the aqueous melatonin composition of claim 1.

11. A method of treating a sleep disorder in a subject, the method comprising administering the aqueous melatonin composition of claim 1 to the subject.

12. The method of claim 11, wherein the sleep disorder comprises insomnia, a sleep disorder associated with the circadian rhythm, delayed sleep phase disorder (DSPS), jet lag, a sleep disorder associated with a psychiatric condition, a sleep disorder associated with neurological disease, a sleep disorder associated with a mental condition, or a sleep disorder associated with a medical disorder.

13. The method of claim 11, wherein the subject is a child or an adolescent.

14. A method for preparing an aqueous melatonin composition, the method comprising:
providing an aqueous phase,
adding potassium sorbate and melatonin to the aqueous phase to obtain an aqueous melatonin solution,
adjusting the pH of said aqueous melatonin solution,
filtering the aqueous melatonin solution,
thereby obtaining said aqueous melatonin composition.

15. An aqueous melatonin composition prepared by the method according to claim 14.

16. The aqueous melatonin composition according to claim 2, wherein the concentration of melatonin is in the range of about 0.9 mg/ml to about 1.1 mg/ml.

17. The aqueous melatonin composition according to claim 4, wherein the concentration of potassium sorbate is in the range of about 0.9 mg/ml to about 1.1 mg/ml.

18. The aqueous melatonin composition according to claim 9, wherein the composition is stable upon storage at room temperature for at least 18 months.

19. The aqueous melatonin composition according to claim 18, wherein the composition is stable upon storage at room temperature for at least 24 months.
